(19) United States
(12) Patent Application Publication
(10) Pub. No.: US 2018/0289641 A1
(43) Pub. Date: Oct. 11, 2018

(54) DEVICES, METHODS, AND COMPOSITIONS FOR CONTROLLING INFECTIONS

(71) Applicant: Innovation Technologies, Inc., Lawrenceville, GA (US)

(72) Inventor: Paul J. RUCINSKI, Ocklawaha, FL (US)

(21) Appl. No.: 16/008,735

(22) Filed: Jun. 14, 2018

Related U.S. Application Data
(63) Continuation of application No. 14/704,224, filed on May 5, 2015, now abandoned.
Application No. 14/704,224 is a continuation of application No. 12/910,820, filed on Oct. 24, 2010, now abandoned.
Application No. 12/910,820 is a continuation-in-part of application No. 12/617,351, filed on Nov. 12, 2009, now abandoned.
Application No. 12/617,351 is a continuation of application No. 11/960,390, filed on Dec. 19, 2007, now Pat. No. 7,662,125.
(60) Provisional application No. 60/875,788, filed on Dec. 19, 2006.
(60) Provisional application No. 61/254,906, filed on Oct. 26, 2009.

(51) Int. Cl. A61K 31/155; A61M 3/02

(52) U.S. Cl. CPC A61K 31/155; A61M 3/02

(57) ABSTRACT

The subject invention provides novel and highly effective methods and devices for convenient and effective wound irrigation.

### CROSS-REFERENCE TO RELATED APPLICATIONS

This application is a continuation of co-pending application Ser. No. 14/704,224, filed May 5, 2015; which is a continuation of application Ser. No. 12/910,820, filed Oct. 24, 2010, which claims the benefit of U.S. Provisional Application Ser. No. 61/254,906, filed Oct. 26, 2009 and which is also a continuation-in-part of U.S. application Ser. No. 12/617,351, filed Nov. 12, 2009; which is a continuation of application Ser. No. 11/960,390, filed Dec. 19, 2007, now U.S. Pat. No. 7,662,125; which claims the benefit of U.S. Provisional Application Ser. No. 60/875,788, filed Dec. 19, 2006, all of which are incorporated herein by reference in their entirety.

### BACKGROUND OF THE INVENTION

In the management and treatment of a wound there are three primary objectives: (1) prevention of infection, (2) preservation and/or restoration of function, and (3) preservation and/or restoration of cosmetic appearance. The most important of these objectives is the prevention of infection. Success in the prevention of infection directly affects the healing process and the degree to which function and cosmetic appearance can be preserved and/or restored. However, heretofore, wound irrigation has not been directly combined with the administration of drugs that can reduce infection or otherwise promote healing.

It is known that the number of bacteria present at the site is a critical determinant of whether a wound becomes infected. Experimental evidence suggests that a critical level of bacteria is approximately 10⁵ organisms per gram of tissue. Below this level, wounds typically heal; at levels greater than 10⁵ bacteria per gram of tissue, wounds often become infected. All traumatic wounds are contaminated by the time the wound is presented to a medical care facility for treatment (Dire, Daniel I. [1990] “A comparison of Wound Irrigation Solutions Used in the Emergency Department,” Annals of Emergency Medicine 19(6):704-708). Dirty wounds, or those that have not been treated within six hours, are likely to be contaminated with bacteria at levels that are higher than the critical level. Reducing the number of bacteria in and around the wound is critical for avoiding infection and expediting wound healing.

Methicillin-resistant Staphylococcus aureus (MRSA) infection is caused by Staphylococcus aureus bacteria—often called “staph.” Decades ago, strains of staph emerged in hospitals that were resistant to the broad-spectrum antibiotics commonly used to treat them. These antibiotics include methicillin and other more common antibiotics such as oxacillin, penicillin and amoxicillin. Dubbed methicillin-resistant Staphylococcus aureus (MRSA), it was one of the first genus to be resistant to all but the most powerful drugs.

Staph bacteria are generally harmless unless they enter the body through a cut or other wound. In older adults and people who are ill or have weakened immune systems, ordinary staph infections can cause serious illness. Staph infections, including MRSA, occur most frequently among persons in hospitals and healthcare facilities, such as nursing homes and dialysis centers, who have weakened immune systems.

In the 1990s, a type of MRSA began appearing in the wider community. Today, that form of staph, known as community-associated MRSA, or CA-MRSA, is responsible for many serious skin and soft tissue infections and for a serious form of pneumonia. When not treated properly, MRSA infection can be fatal.

MRSA infections are spreading rapidly in the United States and worldwide. According to the Center for Disease Control and Prevention (CDC), the proportion of infections that are antimicrobial resistant has been growing. In 1974, MRSA infections accounted for two percent of the total number of staph infections; in 1995 it was 22%; and in 2004 it was nearly 63%. Additionally, recent research has suggested that 30-50% of the population carries MRSA colonies on their bodies all the time, helping to facilitate the spread of infection.

Although MRSA has traditionally been seen as a hospital-associated infection, there has also been an epidemic of CA-MRSA in the United States. MRSA infections in the community are usually manifested as skin infections, such as pimples and boils. These CA-MRSA infections can occur in otherwise healthy people, and commonly occur among athletes who share equipment or personal items including towels and razors. In fact, from 2000 to present, there have been several reported outbreaks of CA-MRSA affecting high school athletic teams. This epidemic among athletes is aided by the fact that MRSA grows very rapidly in warm, moist areas such as gyms and gym locker rooms. Common cuts and abrasions such as those frequently occurring in football and baseball now pose significant threats due to the possibility of an MRSA infection.

Vancomycin is one of the few antibiotics still effective against hospital strains of MRSA infection, although the drug is no longer effective in every case. Several drugs continue to work against CA-MRSA, but CA-MRSA is a rapidly evolving bacterium, and it may be a matter of time before it, too, becomes resistant to most antibiotics.

Chlorhexidine is a chemical antiseptic, and it combats both gram positive and gram negative microbes. It is bacteriostatic, hampering the growth of bacteria, and bacteriocidal, killing bacteria. It is often used as an active ingredient in mouthwash designed to kill dental plaque and other oral bacteria. Chlorhexidine also has non-dental applications. For example, it is used for general skin cleansing, as a surgical scrub, and as a pre-operative skin preparation.

Chlorhexidine is typically used in the form of acetate, gluconate, or hydrochloride, either alone or in combination with other antiseptics such as cetrimide.

### BRIEF SUMMARY OF THE INVENTION

The subject invention provides novel and highly effective methods, devices and compositions for efficient delivery of one or more medications or other active ingredients to a target site in a patient in order to reduce, prevent, and/or treat infection.

Examples of agents that can be administered to a patient in accordance with the subject invention include, but are not limited to, anti-bacterial agents, anti-viral agents, fungicidal agents, chemotherapy agents, topical antiseptics, anesthetic agents, oxygenated fluids and/or agents, antibiotics, diagnostic agents, homeopathic agents, and over the counter medications/agents. In a preferred embodiment, the active agent is chlorhexidine gluconate, preferably at a concentration of less than 1.0%, more preferably less than 0.1% and most preferably at 0.05% or less.

In one embodiment, the subject invention provides a reservoir housing containing an irrigation solution with one or more active agents, wherein the reservoir housing has attached to it a discharge means having one or a plurality of ports through which a sufficient volume of the solution can pass at an appropriate pressure for effective delivery of the solution, including the active agent, to a target site.

### DETAILED DESCRIPTION OF THE INVENTION

The subject invention provides novel, convenient, inexpensive, and effective drug delivery techniques that utilize, in one embodiment, a device having a reservoir housing and a discharge means for delivering an active agent to a target site. The subject invention also provides compositions and methods of use for the device and composition.

The materials and methods of the subject invention make it possible to conveniently and easily apply fluid containing, for example, a medicinal agent to, for example, a wound. In one embodiment the wound is a surgical site.

As used herein, “active agents” refers to compounds or other entities that perform a therapeutic and/or diagnostic function. This function may be direct, such as promoting tissue repair or killing cancer cells, or may be indirect by eliciting a physiological response that ultimately results in the desired beneficial result.

In one embodiment, a sterile water (not saline) solution comprising 0.05% or less (or even less than 0.04% or even less than 0.03%) of chlorhexidine is applied to a wound in the skin of a human. Preferably the wound is then rinsed within five minutes (preferably within 1-3 minutes) with a sterile saline or water liquid that does not contain chlorhexidine.

In another embodiment, the solution is applied to a surgical site.

In a specific embodiment, the chlorhexidine gluconate used according to the subject invention has the following chemical structure:

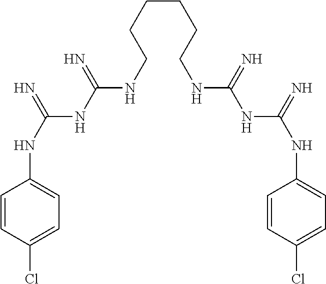 [SMILES: N=C(NCCCCCCNC(=N)NC(=N)Nc1ccc(Cl)cc1)NC(=N)Nc1ccc(Cl)cc1]

Chlorhexidine Gluconate

| | |
|---|---|
| Systematic | 1-[amino-[6-[amino-[amino-(4-chlorophenyl)amino- |
| (IUPAC) Name | methylidene]amino-methylidene]aminohexylimino] |
| | methyl]imino-N-(4-chloropheny1)-methanediamine |

Chemical Data

| | |
|---|---|
| Formula | C₂₂H₃₀Cl₂N₁₀ |
| Mol. weight | 505.446 g/mol |

In a preferred embodiment, about 15-25 mg of chlorhexidine gluconate is applied to a wound. In one embodiment, the wound is an abrasion or laceration and the solution is applied prior to repair/closure.

Preferably, the pH of the solution is neutral or slightly acidic. Preferably the pH is 5.0 to 7.5. More preferably the pH is 5.5. to 7.0. In one embodiment, the chlorhexidine is applied without a sudsing agent.

In certain embodiments, the composition does not have any ingredients that would promote microbial growth. Preferably, the composition does not comprise a consistency builder as that term is used in U.S. Published Application 2007/0184114, which is incorporated herein in its entirety.

In specific embodiments, a CHG-containing solution is applied to an open surgical wound. The solution may be applied under pressure or not under pressure. In one embodiment, the solution is not applied by jet lavage. In a further embodiment, the fluid is not applied under pressure. Preferably, the patient is a human. The patient may also be, for example, a dog, horse, cat, pig or cattle.

Preferably, after administration of the CHG solution, the site is washed with saline within 1, 5, or 10 minutes.

In the context of the operating room, the CHG-containing solution is preferably in a sterile container. The container may be sterilized by, for example, irradiation.

In one embodiment, CHG may be provided in a small ampule such that the contents of the ampule can be added to, for example, a standard sized bottle of water, or to an IV bag or line, to create a CHG solution having a concentration of CHG as described herein.

The solution of the subject invention can also be provided in an IV bag.

In other embodiments, the subject invention provides antimicrobial lotions, creams, sponges, and suppositories. These embodiments preferably contain CHG as the active ingredient. These embodiments provide CHG, at the concentrations set forth herein, directly to a location wherein antimicrobial activity is needed.

The aqueous solution containing CHG may have other components including, for example, pH modifiers, buffers, local anesthetic agents, agents that promote wound healing (such as agents that help degrade biofilm) and other therapeutic and non-therapeutic compounds. In one embodiment, the composition “consists essentially” of an aqueous solution of CHG, which means that the solution contains no other active agent that materially changes the ability of the solution to control bacteria growth.

In a preferred embodiment, the wound is rinsed with saline after application of a CHG-containing solution. Rinsing with saline not only rinses the CHG off but also the saline chemically neutralizes the CHG thereby preventing or reducing edema. The rinse is preferably applied about 30 seconds to about 5 minutes after the administration of the CHG. The rinse may be from about 100 ml to 1000 ml of saline and is typically applied for about 5 seconds to about 1 minute.

In a preferred embodiment, the application of the irrigation solution of the subject invention results in a reduction in the number of bacteria at the wound when compared to either an untreated wound or a wound irrigated with saline or water that does not contain chlorhexidine. Advantageously, the use of CHG according to the subject invention can result in effective control of an infection without causing tissue damage.

Advantageously, the irrigation solution of the subject invention is effective in combating infection, even when organic materials (including blood, tissue, and/or dirt and debris) are present. Of course, such materials are present in all skin wounds.

In addition to killing bacteria, the formulations of the subject invention can also “depathogenize” certain bacteria including, for example, E. coli and Klebsiella aerogenes, making these bacteria less able to cause infection.

Advantageously, the compositions and methods of the subject invention can be used to effectively treat wounds even when biofilm is present.

The drug delivery methods of the subject invention can be used in conjunction with the delivery of an active agent by many of the routes set forth in Table 1. Of particular interest are: cutaneous, intra-abdominal, intracranial, intralesional, intrathoracic (during surgery), irrigation, nasal, in the ear canal, as an oral bowel prep, for gastric lavage, as an eye wash, periodontal, rectal, soft tissue, subcutaneous, and vaginal routes. The compositions of the subject invention can also be used to control acne and other skin infections, including infections of the feet and scalp. In one embodiment, the chlorhexidine composition of the subject invention is used to treat an abscess. Preferably the solution is applied deep into the abscess using, for example, a device with an elongated discharge port to facilitate effective administration.

Under optimal circumstances, the drug delivery devices and methods of the subject invention are utilized by trained medical technicians; however, because of the simplicity and convenience of the devices of the subject invention, they can be used to greatly enhance the effectiveness of drug delivery regardless of the training level of the operator performing the irrigation.

### Delivering Active Agents

Examples of agents that can be administered to a patient in accordance with the subject invention include, but are not limited to, anti-bacterial agents, anti-viral agents, fungicidal agents, chemotherapy agents, topical antiseptics, anesthetic agents, oxygenated fluids and/or agents, antibiotics, diagnostic agents, homeopathic agents, and over-the-counter medications/agents.

The target sites to which an active ingredient can be administered according to the subject invention include, but are not limited to, wounds and surgical sites. The surgical sites may include, for example, joint replacements, abdominal surgery, brain surgery, and oral/periodontal surgery sites. In each case, the ability to deliver the active agent to a specific site, at an appropriate dosage, is unique and highly advantageous.

The solution that carries the active agent can be, for example, water, saline, or a balanced salt solution. The solution is preferably sterile. In the case of CHG, the solution is preferably water. The device can be sterilized by known sterilization techniques, including boiling, autoclaving, gas sterilization, irradiation, and the like, either separately or together with the reservoir housing.

The use of chlorhexidine is particularly advantageous because it is broad spectrum, binds to the skin (to provide residual activity), works rapidly and, when used according to the subject invention, is non-toxic.

Chlorhexidine is a chemical antiseptic that can be used to combat both gram positive and gram negative microbes. It is both bacteriostatic and bactericidal. Various species of bacteria are involved in the pathogenesis of wound infection and/or secondary cellulitis. At times these infections can result in disfigurement, loss of extremities, prolonged convalescences, and/or death. The therapeutic effect of the irrigation solution of the subject invention is to combat microbes typically involved in the pathology of these infections by its antiseptic properties and those associated with the irrigation process itself. Controlling the microbial load in wounds is a vital factor in minimizing infection and thus decreasing and/or preventing disease.

### Spectrum of Activity

Chlorhexidine is active against aerobic and anaerobic gram-positive and gram-negative bacteria. The drug also has some activity against Chlamydia trachomatis, certain fungi, and certain viruses.

### Aerobic Bacteria

Chlorhexidine is highly active against a variety of gram-positive aerobic bacteria, including Streptococcus mutans, S. pyogenes (group A β-hemolytic streptococci), S. salivarius, and S. sanguis . Chlorhexidine is active against Staphylococcus aureus, S. epidermidis, S. haemolyticus, S. hominis, and S. simulans. The drug is active against both oxacillin-resistant (ORSA) and oxacillin-susceptible staphylococci (also known as methicillin-resistant [MRSA] or methicillin-susceptible staphylococci).

Chlorhexidine is active against Enterococcus, including E. faecalis and E. faecium, and is active against both vancomycin-susceptible and vancomycin-resistant strains.

### Anaerobic Bacteria

Chlorhexidine is active against some anaerobic bacteria. The drug is active against some strains of Bacteroides, Propionibacterium, Clostridium difficile, and Selenomonas, but is less active against Veillonella.

### Fungi

Chlorhexidine has some activity against Candida albicans, C. dubliniensis, C. glabrata (formerly Torulopsis glabrata), C. guillermondii, C. kefyr (formerly C. pseudotropicalis), C. krusei, C. lusitaniae, and C. tropicalis (formerly C. parapsilosis). Chlorhexidine also has some activity against dermatophytes, including Epidermophyton floccosum, Microsporum gypseum, M. canis, and Trichophyton mentagrophytes.

### Viruses

Chlorhexidine appears to have antiviral activity against viruses that have a lipid component in their outer coat or have an outer envelope such as cytomegalovirus (CMV), human immunodeficiency virus (HIV), herpes simplex virus types 1 (HSV-1) and 2 (HSV-2), influenza virus, parainfluenza virus, and variola virus (smallpox virus).

### Methods and Formulations

Advantageously, because the methods of the subject invention can be used to accurately and efficiently deliver an active ingredient to a target site in a patient, it is possible, in certain embodiments, to utilize reduced concentrations of active ingredients. Thus, in one embodiment of the subject invention, a low concentration solution of chlorhexidine can be used to effectively reduce infections at, for example, a wound, surgical site, or other tissue opening. In a preferred embodiment, the chlorhexidine solution is less than 4%. In a more preferred embodiment the chlorhexidine is less than 2%, or less than 1% or even less than 0.1%. In one embodiment, the chlorhexidine solution is 0.05%. In a further embodiment, the chlorhexidine solution is between 0.02% and 0.05%. Specifically exemplified herein is the use of chlorhexidine gluconate.

As described above, in one embodiment of the subject invention, the device of the subject invention is used to deliver an active agent, such as an antimicrobial agent, to a target site, such as a wound. Subsequent to the administration of the active agent, the site can then be flushed with, for example, saline to remove at least any excess of the active agent. Preferably, this flushing occurs within five minutes of the administration of the chlorhexidine solution. More preferably, this flushing occurs within one to three minutes of the administration of the chlorhexidine solution. In this way, any potential toxicity associated with the active agent can be reduced or eliminated. In the case of chlorhexidine gluconate, rinsing with an irrigation fluid removes excess chlorhexidine that has not bound to, for example, proteins of the skin.

In yet another embodiment, a diagnostic agent can be administered using the device and method of the subject invention. The diagnostic agent may be, for example, an antibody, protein, or polynucleotide that binds to a target biomolecule. Any such binding may then be visualized utilizing technologies known to those skilled in the art. These technologies include, for example, the use of flourophores or other labels that can be visualized either by the naked eye or through appropriate detection instruments. The diagnostic applications of the subject invention include the detection of bacteria, viruses, parasites and other pathogens. Cancer cells can also be visualized using the diagnostic methods of the subject invention.

In yet another embodiment, the device and method can be used to deliver growth factors and/or protease inhibitors to a target site. Such growth factors and/or protease inhibitors, which can, for example, expedite the healing of wounds, are well known to those skilled in the art.

In yet another embodiment, the method of the subject invention can be used to deliver oxygenated water and/or “enhanced water” to a target site. The enhanced water can be that which is described in, for example, published U.S. Patent Application 20050191364 and the references cited therein (all of which are incorporated herein by reference in their entireties). The use of the subject method for the effective delivery of such oxygenated or enhanced water can be used to promote tissue healing and reduce infections.

In a further embodiment, the device and method of the subject invention can be used to efficiently deliver anti-microbial peptides (AMPs) to a target site. AMPs are well known in the art. Antimicrobial peptides are predominantly small polypeptides that inhibit the growth of microbes. As effectors of innate immunity, antimicrobial peptides directly kill a broad spectrum of bacteria, fungi, and viruses. In addition, these peptides modify the local inflammatory response and activate mechanisms of cellular and adaptive immunity. Cathelicidins and defensins comprise the major families of AMPs in the skin, although other cutaneous peptides, such as proteinase inhibitors, chemokines, and neuropeptides, also demonstrate antimicrobial activity. See, for example, Braff, M. et al., (2005) “Cutaneous Defense Mechanisms by Antimicrobial Peptides,” J Invest Dermatol, 125:9-13.

### The Drug Delivery Device

The device of the subject invention can be, for example, the device as shown in U.S. Published Application No. US-2008-0159963-A1, which is incorporated herein, in its entirety, by reference. The reservoir may be, for example, from 100 ml to 1000 ml.

In one embodiment, the subject invention provides a reservoir housing containing a solution with one or more active agents, wherein the reservoir housing has attached to it a discharge means having one or a plurality of ports through which a sufficient volume of the solution can pass at an appropriate pressure for effective delivery of the solution, including the active agent, to a target site.

The reservoir housing and contents can be stored in a sterile environment, e.g., sterile packaging which is opened immediately prior to use. The reservoir housing can be directed towards the wound and squeezed or compressed to expel or discharge the solution in the desired direction, and at the desired pressure to effect irrigation of a wound to remove contaminants or debris and to deliver the active agent(s).

The discharge means can be packaged separately from the reservoir housing. The discharge means is packaged in a sterile environment. In one embodiment, the drug delivery device is provided in a sterile tray. According to the subject invention, the laceration tray can have, for example, in addition to the drug delivery device of the subject invention, other items conveniently provided for treating wounds. Contemplated items that can be included in a tray include, but are not limited to, needle holders (i.e., 5″ floor-grade smooth); scissors (i.e., 4.5″ floor-grade straight Iris scissors); hemostats (i.e., 5″ floor-grade curved mosquito hemostat); forceps (i.e., floor-grade tissue forceps with 1×2 teeth); cups (i.e., 2 oz. medicine cups); syringes (i.e., 10 cc Luer Lock syringe); needles (i.e., 25 gauge×⅝″ needle; 27 gauge×1.5″ needle; 18 gauge×1.5″ needle); dressings (i.e., gauze dressings); drapes (i.e., polylined fenestrated drapes); and towels (i.e., absorbent towels).

Significantly, it is known that more force is required to rid the wound of particles with a small surface area (e.g., bacteria) than to remove particles with a large surface area (e.g., dirt, sand, or vegetation). Minimum recommended volumes of irrigation solution vary, but for a moderately sized potentially contaminated wound, for example a laceration 3-6 cm long and less than 2 cm deep, at least 200 to 500 ml or more should be used. Greater volumes, on the order of one to two liters, may be required for larger or heavily contaminated wounds. Irrigation should continue at least until all visible, loose particulate matter has been removed.

Following are examples that illustrate procedures for practicing the invention. These examples should not be construed as limiting.

### Example 1—Methods of Irrigation

When a patient presents a wound to a medical or other health care professional skilled in the art, that medical professional assesses the extent of the injury sustained by the patient, including all other life threatening injuries. Appropriate action regarding these life threatening injuries is performed and a history is recorded. All wounds are covered to minimize further contamination until the actual repair process begins.

For examination of the wound, it is assumed that a medical professional would have performed a detailed evaluation of the extent of tissue injury, including but not limited to:

anatomical area considerations, depth of the wound, type of injury, e.g., crash injury, puncture wound, bites, missiles, cuts with sharp objects, or the like. Included in this examination would be a determination of the type(s) of contamination, time elapsed between the occurrence of the injury to presentation, gross contamination of a wound, and other medical factors associated with an increased incidence of infection (for example, diabetics, AIDS patients, and chemotherapeutics patients).

The wound and surrounding tissue, at the option of the health care professional, could be anesthetized using topical, local, or general anesthetics before the wound-cleansing method begins. Alternatively, an anesthetic may be delivered using the device and method of the subject invention.

Manual or mechanically produced pressure is applied to the reservoir housing to expel the irrigation solution with active agent through the nozzles of the discharge means. The wound should be irrigated in this fashion until all visible evidence of contamination has been removed. A potentially contaminated wound of any size should be irrigated with a minimum of 200-300 ml of irrigation solution. Heavily contaminated or larger wounds may require 2-3 liters of irrigation solution.

Following an initial irrigation of the wound, a re-examination of the wound should be undertaken. The wound should be explored to its base to ascertain that no visible foreign bodies or contaminants remain. If foreign bodies or contaminants are found, the irrigation process should be repeated followed by a re-examination. This may continue for several cycles. Once irrigation has been completed, i.e., no visible contaminants remain, the damaged tissue would be repaired in a standard accepted fashion.

Irrigation of skin wounds such as cuts, scrapes, punctures, abrasions, etc. are particularly well-suited for irrigation according to the subject invention, as is irrigation of surgical sites.

### Example 2—Routes of Administration

Table 1 provides a listing of various routes of administration that can be used according to the subject invention.

TABLE 1
Routes of Administration

| Delivery Route | Description |
|---|---|
| AURICULAR (OTIC) | Administration to or by way of the ear. |
| BUCCAL | Administration directed toward the cheek, generally from |
| | within the mouth. |
| CONJUNCTIVAL | Administration to the conjunctiva, the delicate membrane |
| | that lines the eyelids and covers the exposed surface of the |
| | eyeball. |
| CUTANEOUS | Administration to the skin. |
| DENTAL | Administration to a tooth or teeth. |
| ENDOCERVICAL | Administration within the canal of the cervix uteri. |
| | Synonymous with the term intracervical.. |
| ENDOSINUSIAL | Administration within the nasal sinuses of the head. |
| ENDOTRACHEAL | Administration directly into the trachea. |
| ENTERAL | Administration directly into the intestines. |
| EPIDURAL | Administration upon or over the dura mater. |
| EXTRA-AMNIOTIC | Administration to the outside of the membrane enveloping |
| | the fetus |
| EXTRACORPOREAL | Administration outside of the body. |
| INFILTRATION | Administration that results in substances passing into tissue |
| | spaces or into cells. |
| INTERSTITIAL | Administration to or in the interstices of a tissue. |
| INTRA-ABDOMINAL | Administration within the abdomen. |
| INTRA-ARTICULAR | Administration within a joint. |
| INTRABILIARY | Administration within the bile, bile ducts or gallbladder. |
| INTRABRONCHIAL | Administration within a bronchus. |
| INTRACARDIAC | Administration with the heart. |
| INTRACARTILAGINOUS | Administration within a cartilage; endochondral. |
| INTRACAVERNOUS | Administration within a pathologic cavity, such as occurs in |
| | the lung in tuberculosis. |
| INTRACAVITARY | Administration within a non-pathologic cavity, such as that |
| | of the cervix, uterus, or penis, or such as that which is |
| | formed as the result of a wound. |
| INTRACEREBRAL | Administration within the cerebrum. |
| INTRACORPORUS CAVERNOSUM | Administration within the dilatable spaces of the corporus |
| | cavernosa of the penis. |
| INTRADUCTAL | Administration within the duct of a gland. |
| INTRADUODENAL | Administration within the duodenum. |
| INTRADURAL | Administration within or beneath the dura. |
| INTRAESOPHAGEAL | Administration within the esophagus. |
| INTRAGASTRIC | Administration within the stomach. |
| INTRAILEAL | Administration within the distal portion of the small |
| | intestine, from the jejunum to the cecum. |
| INTRALESIONAL | Administration within or introduced directly into a localized |
| | lesion. |
| INTRALUMINAL | Administration within the lumen of a tube. |
| INTRALYMPHATIC | Administration within the lymph. |
| INTRAMEDULLARY | Administration within the marrow cavity of a bone. |
| INTRAMENINGEAL | Administration within the meninges (the three membranes |
| | that envelope the brain and spinal cord). |
| INTRAOCULAR | Administration within the eye. |
| INTRAOVARIAN | Administration within the ovary. |
| INTRAPERICARDIAL | Administration within the pericardium. |
| INTRAPERITONEAL | Administration within the peritoneal cavity. |
| INTRAPLEURAL | Administration within the pleura. |
| INTRAPROSTATIC | Administration within the prostate gland. |
| INTRAPULMONARY | Administration within the lungs or its bronchi. |
| INTRASINAL | Administration within the nasal or periorbital sinuses. |
| INTRASPINAL | Administration within the vertebral column. |
| INTRASYNOVIAL | Administration within the synovial cavity of a joint. |
| INTRATENDINOUS | Administration within a tendon. |
| INTRATESTICULAR | Administration within the testicle. |
| INTRATHECAL | Administration within the cerebrospinal fluid at any level of |
| | the cerebrospinal axis, including injection into the cerebral |
| | ventricles. |
| INTRATHORACIC | Administration within the thorax (internal to the ribs); |
| | synonymous with the term endothoracic. |
| INTRATUBULAR | Administration within the tubules of an organ. |
| INTRATUMOR | Administration within a tumor. |
| INTRATYMPANIC | Administration within the aurus media. |
| INTRAUTERINE | Administration within the uterus. |
| INTRAVESICAL | Administration within the bladder. |
| INTRAVITREAL | Administration within the vitreous body of the eye. |
| IRRIGATION | Administration to bathe or flush open wounds or body |
| | cavities. |
| LARYNGEAL | Administration directly upon the larynx. |
| NASAL | Administration to the nose; administered by way of the nose. |
| NASOGASTRIC | Administration through the nose and into the stomach, |
| | usually by means of a tube. |
| OCCLUSIVE DRESSING | Administration by the topical route which is then covered by |
| TECHNIQUE | a dressing which occludes the area. |
| OPHTHALMIC | Administration to the external eye. |
| ORAL | Administration to or by way of the mouth. |
| OROPHARYNGEAL | Administration directly to the mouth and pharynx. |
| PERCUTANEOUS | Administration through the skin. |
| PERIARTICULAR | Administration around a joint. |
| PERIDURAL | Administration to the outside of the dura mater of the spinal |
| | cord.. |
| PERINEURAL | Administration surrounding a nerve or nerves. |
| PERIODONTAL | Administration around a tooth. |
| RECTAL | Administration to the rectum. |
| RESPIRATORY (INHALATION) | Administration within the respiratory tract by inhaling orally |
| | or nasally for local or systemic effect. |
| RETROBULBAR | Administration behind the pons or behind the eyeball. |
| SOFT TISSUE | Administration into any soft tissue. |
| SUBARACHNOID | Administration beneath the arachnoid. |
| SUBCONJUNCTIVAL | Administration beneath the conjunctiva. |
| SUBCUTANEOUS | Administration beneath the skin; hypodermic. Synonymous |
| | with the term SUBDERMAL. |
| SUBLINGUAL | Administration beneath the tongue. |
| SUBMUCOSAL | Administration beneath the mucous membrane. |
| TOPICAL | Administration to a particular spot on the outer surface of the |
| | body. The E2B term TRANSMAMMARY is a subset |
| | of the term. |
| TRANSMUCOSAL | Administration across the mucosa. |
| TRANSPLACENTAL | Administration through or across the placenta. |
| TRANSTRACHEAL | Administration through the wall of the trachea. |
| TRANSTYMPANIC | Administration across or through the tympanic cavity. |
| URETERAL | Administration into the ureter. |
| URETHRAL | Administration into the urethra. |
| VAGINAL | Administration into the vagina. |

It should be understood that the examples and embodiments described herein are for illustrative purposes only and that various modifications or changes in light thereof will be suggested to persons skilled in the art and are to be included within the spirit and purview of this application and the scope of the appended claims.

I claim:

1. A sterile container having contained therein a sterile aqueous solution comprising chlorhexidine at a concentration of 0.05% or less.

2. The container, according to claim 1, which contains from 100 ml to 1000 ml of said solution.

3. The container, according to claim 1, wherein said container further comprises a backsplash shield.

4. The container, according to claim 1, wherein the chlorhexidine is present at a concentration of 0.05%.

5. The container, according to claim 1, wherein the chlorhexidine is in the form of chlorhexidine gluconate.

6. A method for irrigating a wound, said method comprising applying the contents of the container of claim 1 to a wound.

7. The method, according to claim 6, wherein said device further comprises a backsplash shield.

8. The method, according to claim 6, further comprising rinsing the wound within 5 minutes with a solution that does not contain chlorhexidine.

9. The method, according to claim 6, used to treat a wound in a human.

10. The method, according to claim 6, wherein the chlorhexidine is at a concentration of 0.05%.

11. The method, according to claim 6, used to irrigate a surgery site.

12. The method, according to claim 11, where the type of surgery is selected from the group consisting of orthopedic, cardiac, brain, and abdominal surgeries.

13. The method, according to claim 6, used to treat a MRSA infection.

14. The method, according to claim 13, wherein the presence of MRSA is known prior to the administration of the solution.

15. The method, according to claim 6, used to treat an abscess.

16. An ampule containing a solution of chlorhexidine at a concentration such that, when the contents of the ampule is mixed with a known volume of water ranging from 250 ml to 5 liters, the resulting solution has a chlorhexidine concentration of less than 1.0%.

17. A cream, lotion, sponge, suppository, or coated indwelling medical device that delivers, to a site of infection, chlorhexidine at a concentration of 0.05% or less.

18. Use of a composition of claim 17 to control an infection.

19. A kit comprising a container according to claim 1 as well as a container of saline.

20. The kit, according to claim 19, which is sterile.
